(19) United States
(12) Patent Application Publication
(10) Pub. No.: US 2025/0387180 A1
(43) Pub. Date: Dec. 25, 2025

(54) SURGICAL ROBOTIC INSTRUMENT EXCHANGE

(71) Applicant: KARL STORZ SE & Co. KG, Tuttlingen (DE)

(72) Inventors: Caleb Thomas Osborne, Hillsborough, NC (US); Kevin Andrew Hufford, Durham, NC (US); Matthew David Marchese, Cary, NC (US); Jonah Kadoko, Cary, NC (US); Mark J. McCue, Durham, NC (US)

(73) Assignee: KARL STORZ SE & Co. KG, Tuttlingen (DE)

(21) Appl. No.: 19/304,443

(22) Filed: Aug. 19, 2025

Related U.S. Application Data
(63) Continuation of application No. PCT/US2024/016579, filed on Feb. 20, 2024.
(60) Provisional application No. 63/485,986, filed on Feb. 20, 2023.

(51) Int. Cl. A61B 34/30; A61B 17/00

(52) U.S. Cl. CPC A61B 34/30; A61B 17/00; A61B 2017/00057; A61B 2017/0046

(57) ABSTRACT

A system and method for facilitating removal of a surgical instrument from a robotic manipulator provides a surgical instrument having an instrument adapter that is mountable to an expandable receiver of a surgical robotic system. With the instrument adapter positioned in the receiver, the user grasps the surgical instrument, pressing one or more exchange members, causing them to change position relative to the receiver. The exchange members are configured so that their position will change relative to the receiver only if the user has a firm grasp on the adapter. The receiver detects the change in position of the exchange members and, in response, moves the receiver from the closed position to the open position so the user can exchange the instrument.

### RELATED APPLICATIONS

This application is a continuation of International Application No. PCT/US2024/016579, filed on Feb. 20, 2024, which claims priority to U.S. Provisional Application No. 63/485,986, filed Feb. 20, 2023.

### TECHNICAL FIELD OF THE INVENTION

The present invention relates generally to the field of surgical devices and systems, and more particularly to the field of surgical instruments for robot-assisted surgery.

### BACKGROUND

There are various types of surgical robotic systems on the market or under development. Some surgical robotic systems use a plurality of robotic manipulators or arms. Each arm carries a surgical instrument, or the camera used to capture images from within the body for display on a monitor. Typical configurations allow two or three instruments and the camera to be supported and manipulated by the system. Input to the system is generated based on input from a surgeon positioned at a surgeon console, typically using input devices such as input handles. The system responds to movement of a user input device by controlling the robotic manipulator that is associated with that input device to position, orient, and actuate the surgical instrument on that manipulator. The image captured by the camera is shown on a display at the surgeon console. The console may be located patient-side, within the sterile field, or outside of the sterile field.

The robotic arms/manipulators include a portion, typically at the terminal end of the arm, that is designed to support and operate a surgical device assembly. The surgical device assembly includes a surgical instrument having a shaft and a distal end effector on the shaft. The end effector is positionable within a patient. The end effector may be one of many different types that are used in surgery including, without limitation, end effectors having one or more of the following features: jaws that open and close, a section at the distal end of the shaft that bends or articulates in one or more degrees of freedom, a tip that rolls axially relative to the shaft, a shaft that rolls axially relative to the manipulator arm.

In many robotic surgical systems, including those using rigid shaft instruments, the surgical instruments are both robotically manipulated by the robotic manipulator arms disposed outside the patient's body, as well as electromechanically actuated within the patient's body. In many surgical systems, robotic manipulation pivots the instrument shaft relative to the patient and may alter the insertion depth of the instrument and/or cause the instrument to roll about its longitudinal axis. Additionally, electromechanical actuation (or hydraulic/pneumatic actuation) may open and close jaws of the instrument, and/or actuate articulating or bending of the distal end of the instrument shaft, and/or roll the instrument's shaft or distal tip. Some systems may use only this latter form of instrument motion while holding the more proximal part of the instrument in a fixed position outside the body using a fixed support or inactive robotic manipulator.

A proximal housing is typically positioned on the proximal end of the instrument shaft. This housing functions as an adapter or interface between the surgical instrument and the robotic manipulator arm. The adapter may include passive actuation mechanisms that receive motion transferred from active actuators in the robotic manipulator or other support to drive functions of the instrument end effector. Such functions may include jaw open-close, shaft articulating or bending, or other functions. As noted above, the instrument actuators for driving the motion of the end effector, which respond to user input to cause actuation of the instrument's functions, are electromechanical motors or other types of actuators. They are often positioned in the terminal portion of the robotic manipulator. In some cases, they are positioned in the proximal housing of the surgical device assembly. In still other configurations some are in the proximal housing while others are in the robotic manipulator. In the latter example, some functions of the end effector might be driven using one or more motors in the terminal portion of the manipulator while other motion might be driven using motors in the proximal housing. See, for example, US 2016/0058513, Surgical System with Sterile Wrappings, in which jaw open-close functions are initiated using electromechanical actuators in the robotic manipulator, and in which rotation or swivel functions of the instrument are initiated using electromechanical actuators housed in the proximal housing of the surgical device assembly.

In some systems, some of the surgical instruments may be removably connected to their respective adapters. This facilitates post-surgery cleaning and sterilization of the instruments and adapters, by allowing them to be separated.

For surgical instruments that are actuated to carry out jaw open-close, shaft articulating or bending, or other functions, there is typically a mechanical interface between the adapter and the robotic manipulator through which motion generated by the instrument actuators within the robotic manipulator is communicated to one or more mechanical inputs of the adapter to control any degrees of freedom of the instrument and, if applicable, its jaw open-close function. This motion may be communicated through a drape positioned between the sterile adapter and the non-sterile manipulator arm. In some commercially available robotic systems, the motion is communicated using rotary connections in which rotating disks on the manipulator transfer motion to rotating disks on an instrument adapter. See, for example, the configuration shown in U.S. Pat. No. 6,491,701. In the embodiment shown in U.S. Pat. No. 9,358,682,a transverse slider pin extends laterally from one side of the case mounted to the proximal end of the instrument. It is moveable to open and close jaws of the instrument (FIG. 18 of the patent). When the instrument is mounted to the manipulator arm, the slider pin is received by a corresponding component 430 (FIG. 19) in the manipulator arm. When it is necessary to open/close the instrument jaws, the component 430 is translated on a carriage by motors in the laparoscopic instrument actuator 400 of the manipulator arm. This advances the slider pin 314 to actuate the jaws.

The instruments are exchangeable during the course of the procedure, allowing one instrument (with its corresponding adapter) to be removed from a manipulator and replaced with another. To minimize surgical procedure time and make efficient use of operating room personnel, it is essential to make the instrument exchange process as efficient as possible.

For robust, safe instrument exchange, it is important to both ensure that the user has a secure hold on the instrument and is also in a safe position relative to a moving robotic manipulator arm.

Applicant's co-pending application published as US 2021/169595, which is incorporated herein by reference, describes a compact actuation assembly, also referred to an an instrument assembly or “IDS”, that is expandable to receive an adapter positioned at the proximal end of a surgical instrument, and that assumes a closed position to engage the instrument so it can be both maneuvered by the robotic manipulator and actuated as needed for jaw open-close, shaft articulating or bending or other functions. Instrument exchange using that configuration involves opening the IDS so that the existing instrument can be removed. The proximal end adapter of the replacement instrument is then positioned, and the IDS is moved to the closed position to engage and actuate in.

When a user wishes to change one surgical instrument held by the robotic manipulator for another surgical instrument, it is necessary to retract the surgical instrument out of and away from the body of the patient. This is typically performed in a handguiding operation in which the user uses his or her hands to retract the manipulator arm so the instrument passes out of and away from the body.

In prior art systems, a distal linear rail is used to provide translation of a surgical instrument along its longitudinal axis, which may be referred to as the z-axis. This linear rail provides predictable motion of the instrument during instrument exchange.

The present application provides for linearly-constrained motion during an instrument exchange without requiring the physical linear rail.

### BRIEF DESCRIPTION OF THE DRAWINGS

FIG. 1 is a perspective view of a robot-assisted surgical system with which the configurations described herein may be utilized;

FIG. 2 is a perspective view of a robotic manipulator arm with the receiver/IDS and instrument assembly mounted to it;

FIG. 3 are a perspective view showing the receiver of FIG. 2 and the surgical instrument separated from the receiver;

FIG. 4 is a perspective view showing the instrument mounted to the IDS;

FIG. 5A is similar to FIG. 4, but shows a sterile drape between the instrument adapter and IDS;

FIG. 5B shows the IDS in the expanded position, with the drape in position. The drape connector is shown separated from the drape so the features within the IDS that receive the drape connector are visible;

FIG. 6 is a perspective view showing an instrument adapter equipped with exchange levers;

FIG. 7A is a perspective view of an IDS and FIG. 7B is a plan view of the distal end of the IDS, each shows the detectors;

FIG. 8 is a perspective view of an IDS, with several features omitted. The IDS housing is shown as transparent view to permit internal features to be visible.

FIG. 9 is a section view of an instrument adapter attached to an IDS, denoting components associated with instrument exchange;

FIGS. 10A and 10B are diagrams of the rear of the instrument adapter, showing the motion of the detection target when the User Press Region of the instrument exchange levers are pressed.

FIGS. 11A and 11B illustrate instrument usage state and instrument exchange requested states for an IDS in which detection is performed using optical sensors;

FIG. 12 is a perspective view of one example of a type of sensor that may be used in the configuration shown in FIGS. 11A and 11B;

FIGS. 13A and 13B illustrate instrument usage state and instrument exchange requested states for an IDS in which detection is performed using optical sensors;

FIG. 13C is similar to FIG. 13B and illustrates the use of the exchange levers for orientation sensor. FIG. 13D is a partially exploded view of an exchange lever of FIG. 13C.

FIG. 14 illustrates a method of using the described features;

FIGS. 15A and 15B schematically illustrate an alternative embodiment incorporating a camming feature for camming the instrument adapter apart from the drape connector.

### DETAILED DESCRIPTION

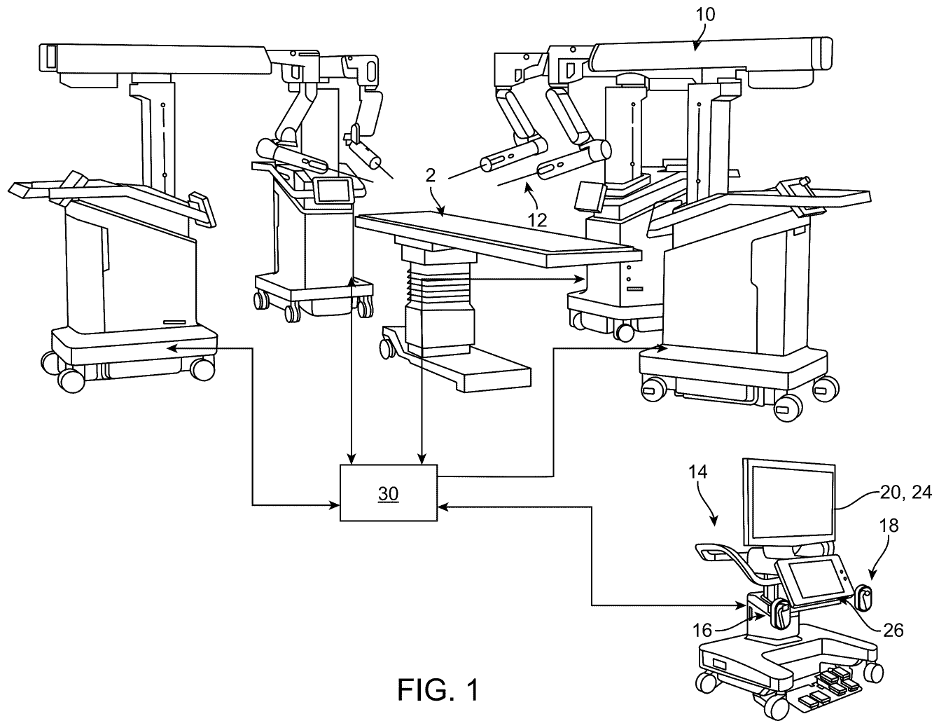

Although the concepts described herein may be used on a variety of robotic surgical systems, the embodiments will be described with reference to a system of the type shown in FIG. 1. In the illustrated system, robotic manipulators 10 are disposed adjacent to a patient bed 2. Each manipulator 10 is configured to maneuver a surgical instrument 12 which has a distal end effector positionable in a patient body cavity. FIG. 1 shows four robotic manipulators, although in other configurations, the number of manipulators may differ.

A surgeon console 14 has two input devices such as handles 16, 18. The input devices are configured to be manipulated by a user to generate signals that are used to command motion of the robotic manipulators in multiple degrees of freedom in order to maneuver the instrument end effectors within the body cavity. The input devices may be mounted to linkages, gimbals, etc. equipped with sensors that generate signals corresponding to positions or movement of the input devices in manners known to those skilled in the art. In other embodiments, the input devices may take the form of handles that are tracked using a tracking system, such as an optical tracking system or an electromagnetic tracking system, either alone or in combination with other sensors within the handles, such as IMUs etc.

In use, a user selectively assigns the two handles 16, 18 to two of the robotic manipulators 10, allowing surgeon control of two of the surgical instruments 12 at any given time. To control a third one of the instruments disposed at the working site, one of the two handles 16, 18 may be operatively disengaged from one of the initial two instruments and then operatively paired with the third instrument, or another form of input may control the third instrument as described in the next paragraph.

One of the instruments 12 is a camera that captures images of the operative field in the body cavity. The camera may be moved by its corresponding robotic manipulator using input from a variety of types of input devices, including, without limitation, one of the handles 16, 18, additional controls on the console, a foot pedal, an eye tracker 20, voice controller, etc. The console may also include a display or monitor 24 configured to display the images captured by the camera, and for optionally displaying system information, patient information, etc. An auxiliary display 26, which may be a touch screen display, can further facilitate interactions with the system.

The surgical system allows the operating room staff to remove and replace the surgical instrument 12 carried by a robotic manipulator 10, based on the surgical need. When an instrument exchange is necessary, surgical personnel remove an instrument from a manipulator arm and replace it with another.

As discussed, manipulation of the input devices 16, 18 results in signals that are processed by the system to generate instructions for commanding motion of the manipulators in order to move the instruments in multiple degrees of freedom and to, as appropriate, control operation of electromechanical actuators/motors that drive instrument functions such as articulation, bending, and/or actuation of the instrument end effectors. One or more control units 30 are operationally connected to the robotic arms and to the user interface. The control units receive user input that is generated as a result of movement of the input devices, and generates commands for the robotic arms to manipulate the surgical instruments so that the surgical instruments are positioned and oriented in accordance with the input provided by the user.

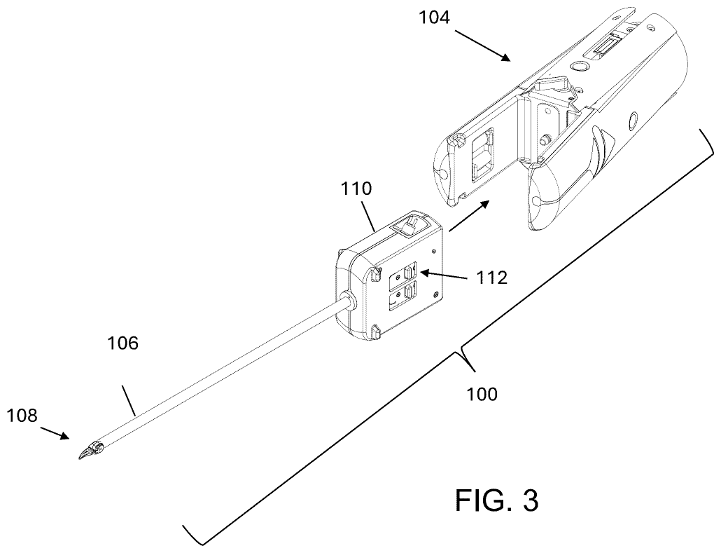

Referring to FIG. 3, positioned at the distal end of each manipulator arm is a receiver 104, which may also be referred to as an instrument drive assembly (IDS). A different surgical instrument 12 is removably mountable to each IDS. As best seen in FIG. 3, each instrument 12 includes an elongate shaft 106, which is preferably rigid but which may be flexible or partially flexible in alternative systems. An end effector 108 is positioned at the distal end of shaft 106, and a base assembly or adapter assembly 110 is at the proximal end.

Instrument and IDS configurations suitable for use with the disclosed inventions will next be described, but it should be understood that these are given by way of example only. The disclosed manipulator may be used with various configurations of instruments and instrument drive systems. More particularly, while the receiver/IDS described here is configured to drive pitch and jaw motion of an articulated surgical instrument, in alternative embodiments the receiver/IDS may have less functionality. In some alternative configurations, it may serve simply to receive an instrument and to drive jaw open/close operations. In other configurations, it may be configured, along with the instrument, to actuate a roll function of the instrument tip relative to the shaft of the instrument.

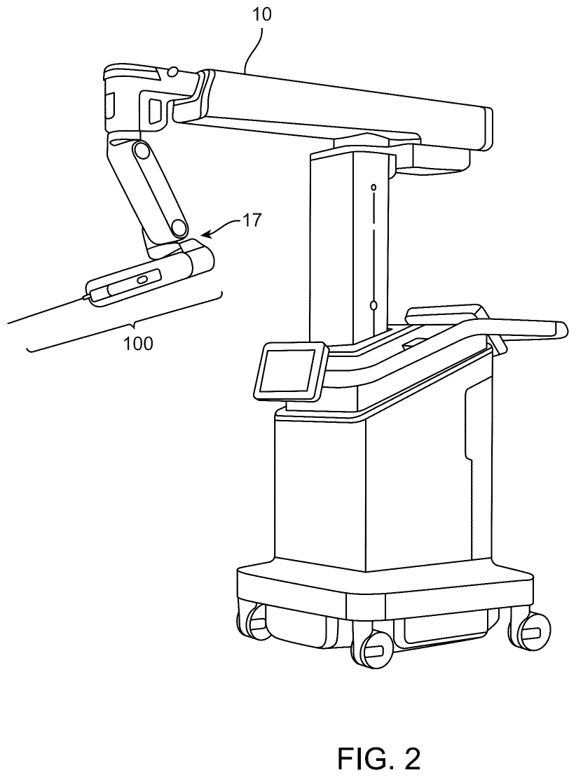

The instrument depicted in the drawings is the type described in Applicant's commonly-owned co-pending application published as US 2020/0375680, entitled Articulating Surgical Instrument, which is incorporated herein by reference. It makes use of four drive cables two of which terminate at one of the jaw members and the other two of which terminate at the other jaw member. This can be two cables looped at the end effector (so each of the two free ends of each cable loop is at the proximal end) or it can be four individual cables. As described in the co-pending application, the tension on the cables is varied in different combinations to effect pitch and yaw motion of the jaw members and jaw open-close functions. Other instruments useful with the system will have other numbers of cables, with the specific number dictated by the instrument functions, the degrees of freedom of the instrument and the specific configuration of the actuation components of the instrument. Note that in this description the terms “tendon,” “wire,” and “cable” are used broadly to encompass any type of tendon that can be used for the described purpose. The surgical instrument's drive cables extend from the end effector 108 through the shaft 106 (FIG. 2) and extend into the adapter assembly 110 where they are coupled to mechanical actuators. A more detailed description is given in Applicant's co-pending application published as US 2021/169595, which is incorporated herein by reference, but a general configuration of these actuators with respect to the adapter assembly will be provided here.

The adapter assembly 110 (which will also be referred to as the “adapter”) may include an enclosed or partially enclosed structure such as a housing or box, or it may be a frame or plate. The exemplary adapter 110 shown in the drawings includes mechanical input actuators 112 exposed to the exterior of the surgical instrument 102. In FIG. 3, two mechanical input actuators 112 are exposed at a first lateral face of the adapter 110. A second two mechanical input actuators 112 (not visible in FIG. 3) may be exposed at the second, opposite, lateral face of the adapter 110, preferably but optionally in a configuration identical or similar to the configuration shown in FIG. 3.

Each of the mechanical input actuators 112 is moveable relative to the adapter 110 between first and second positions. In the specific configuration shown in the drawings, the actuators are longitudinally moveable relative to the housing between a first (more distal) position and a second (more proximal) position such as that shown in FIG. 3. The direction of motion, however, is not required to be longitudinal and can extend in any direction.

In this configuration, the adapter thus has four drive inputs, one for each of the input actuators 112, exposed to its exterior. The illustrated adapter has two parallel planar faces, with two of these inputs positioned on each of the faces. While it may be preferred to include the inputs on opposite sides of the proximal body, other arrangements of inputs on multiple faces of the proximal body can instead be used. Each of these configurations advantageously arranges the drive inputs to maximize the distance between control inputs, minimizing stresses in the sterile drape that, in use, is positioned between the proximal body and the receiver 104. Co-pending US 2021/169595 includes further description of the adapter shown in FIG. 3.

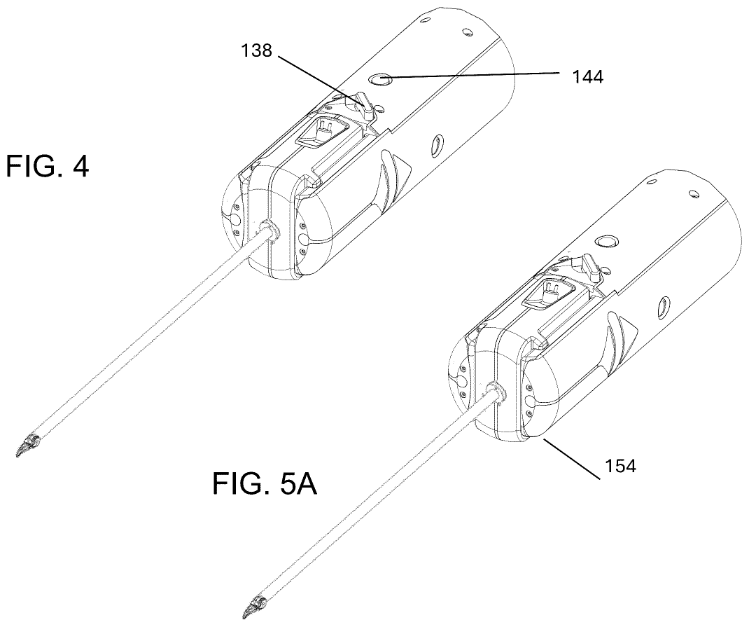

The IDS 104 at the end of each manipulator 10 has an open position (shown in FIG. 3) in which it removably receives the adapter 110 of a corresponding instrument 12, to form an assembly 100. After the adapter 110 is placed within the IDS, the IDS is moved to the closed position shown in FIG. 4, capturing the adapter 110. In this position, the drive inputs 112 of the adapter can engage with corresponding drive outputs 114 of the IDS. As described in detail in co-pending US 2021/169595, user input at the input devices 16, 18 commanding jaw open-close, pitch or yaw articulation etc. of the instrument causes electromechanical actuators in the IDS to move the drive outputs 114. The motion of those drive outputs moves corresponding ones of the adapter's drive inputs 112, altering tension on the instrument's drive cables in a manner that causes the desired motion at the instrument's end effector.

It is important to know that the IDS described in this application and in US2021/169595 are examples of the types of instrument drive systems that can make use of the inventive concepts described below. However, these concepts are equally suitable to any system in which an instrument exchange requires release of the instrument's adapter from a support in a manner that necessitates that the system receive confirmation that the user is supporting the instrument before the instrument will be released.

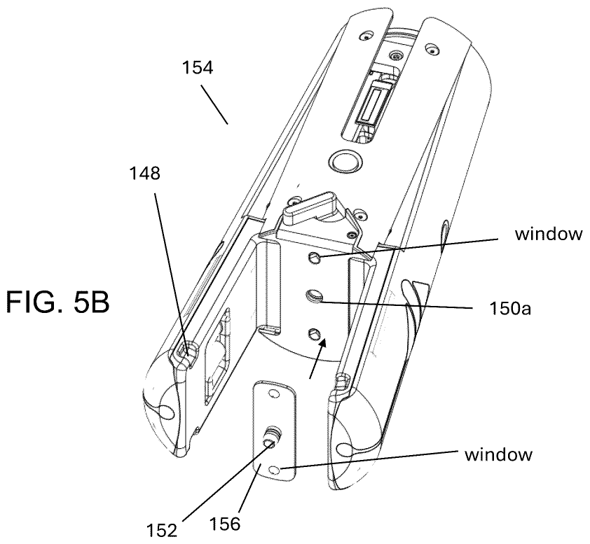

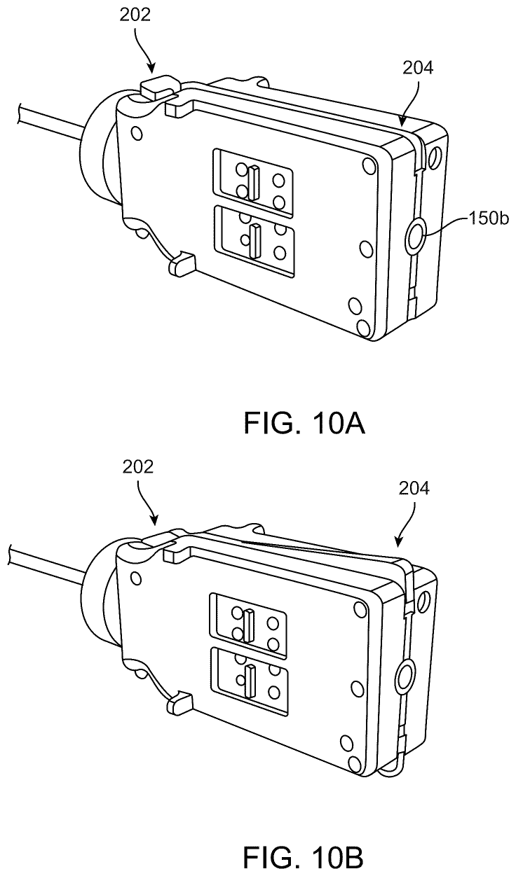

The IDS and robotic manipulator are typically non-sterile components that are covered by a sterile drape before attachment of the sterile surgical instrument. FIG. 5 shows the IDS engaged with an instrument with the drape 154 or barrier between them. As described previously, at the interface between the drive elements of the IDS and the driven elements of the adapter, the end effector motion described above is communicated through the drape to control the degrees of freedom of the instrument. The drape may include an embedded plastic “drape connector” 156 adhered such that the connector has geometry extending to both sides of the drape. For example, the drape connector includes mating pins, posts, conical elements etc. 152 in the proximally-facing direction that mate with female parts 150a (e.g., recesses, conical divots, holes or similar alignment features) in the seat of the IDS 104, and identical or similar elements on the distally-facing face that mate with the female parts 150b on the instrument adapter. See FIGS. 5B and 10A.

The robotic manipulator may be one that robotically manipulates the instrument 102 in one or more degrees of freedom during a procedure (such as the type shown in FIG. 1), or a support that remains stationary during the course of surgery embodiments of a surgical instrument for a robotic surgical system. As described in detail in co-pending US 2021/169595, user input at the input devices 17, 18 commanding jaw open-close, pitch or yaw articulation etc. of the instrument causes electromechanical actuators in the receiver 104 of the arm 14 to move drive elements in the receiver 104. The motion of those drive members moves corresponding ones of the adapter's actuators 112, altering tension on the instrument's drive cables in a manner that causes the desired motion at the instrument's end effector.

### Communicating User Intent and Instrument Security During Instrument Removal

This section describes the features that improve the ease of use of the IDS and adapter described in US 2021/169595. In general, these features perform one or any combination of the following functions:
(a) provide a mechanism by which system will allow or initiate opening of the IDS, or release of an instrument from the IDS, only once the system receives signals confirming that the user is engaging the adapter with his or her hand with sufficient force to prevent dropping of the instrument when the IDS is opened, or the instrument released;
(b) provide a mechanism by which the user can give input instructing the system to open the IDS, release the instrument from the IDS, or initiate some other action related to removal of the instrument from the IDS. This input can additionally give rise to other responses as well, such as withdrawal of the IDS away from the adapter (z-axis withdrawal) using the joints of the robotic manipulator;
(c) preferably carry out the functions described in (a) and (b) with a single action on the part of the user. In such embodiments, the user may simply grasp the adapter with one hand, and this sole action will cause the system to generate signals that both confirm the user is grasping the adapter and that initiate the opening of the IDS or release of the IDS from the system. However, in other embodiments, the user actions causing functions (a) and (b) might be separate actions (e.g., grasp the adapter to generate signals that confirm the user is grasping the adapter, and take another action such as pressing a button, turning a lever, etc. while simultaneously maintaining a grasp on the instrument, or order to initiate the opening of the IDS or release of the IDS from the system).

### Structure

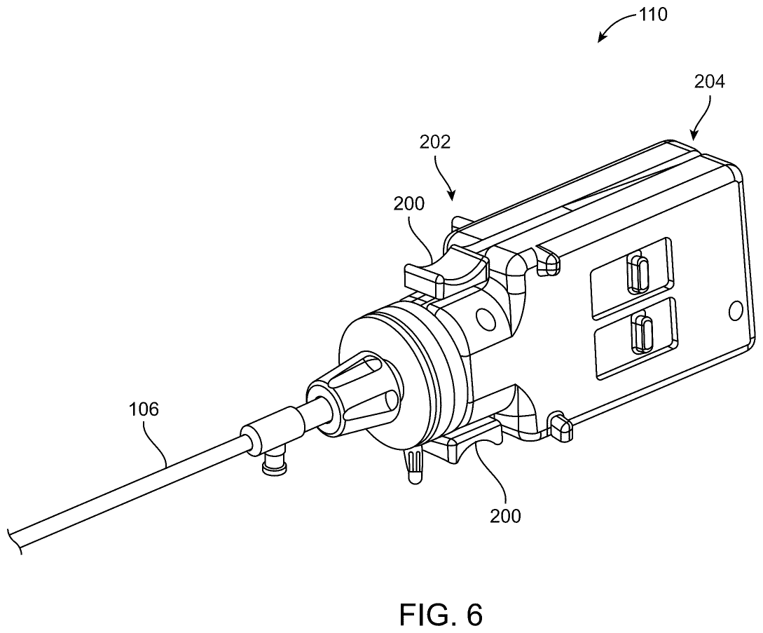

Referring to FIG. 6, in an exemplary embodiment of the present invention, exchange input assemblies are provided on the instrument adapter. The exchange input assemblies are mechanical features that a user engages to generate an electronic signal indicating that the user wishes to remove the instrument and is grasping the adapter. The exchange input assemblies illustrated in the drawings are a pair of exchange members or levers 200 on the instrument adapter. Each exchange lever 200 includes a distal part 202 configured to be pressed by a user, and to advance inwardly towards the longitudinal axis of the adapter when pressed. The distal part 202 may be shaped to receive the user's fingers, forming a defined press region or button as shown.

The exchange lever 200 further includes a proximal part 204 that is displaced when the user presses the distal part. In the embodiments shown in the drawings, the exchange lever pivots around a pivot point or hinge disposed between the proximal and distal parts, such that when the distal part 202 is depressed, the proximal part 204 pivots away from the longitudinal axis of the adapter. As best seen in FIG. 8, a detection target 206 is positioned on the proximal part of the exchange lever 200.

It should be noted that the specific mechanical features of the exchange input assembly can vary between embodiments, and this description should not be interpreted as limiting the invention to any specific configuration of exchange input assembly or exchange lever. Any structure or assembly configured to produce similar action as the exchange levers described in this application, i.e., mechanical displacement of one or more detection targets relative to corresponding detectors—discussed below—in response to a user grasping the instrument adapter, can be used instead. In some variations the exchange input assembly may comprise an assembly or two or more links, flexures, or other components operatively associated to physically displace detection targets in response to a user grasping of the instrument. Mechanisms can be used that pivot around a pivot or hinge (or set of hinges/pivots), the mechanisms might be flexures or sets of flexures. Where flexures are used, they may be integrated as a portion of corresponding exchange levers, or they might be multi-part components. In still other implementations, the instrument exchange assembly may comprise a pair of mechanical switches or buttons not connected to a pivoting or moving linkage or lever. In such implementations, detection that both buttons/switches have been depressed at the same time signals to the system indicating that user is grasping the adapter and wishes to remove the instrument from the IDS exchange.

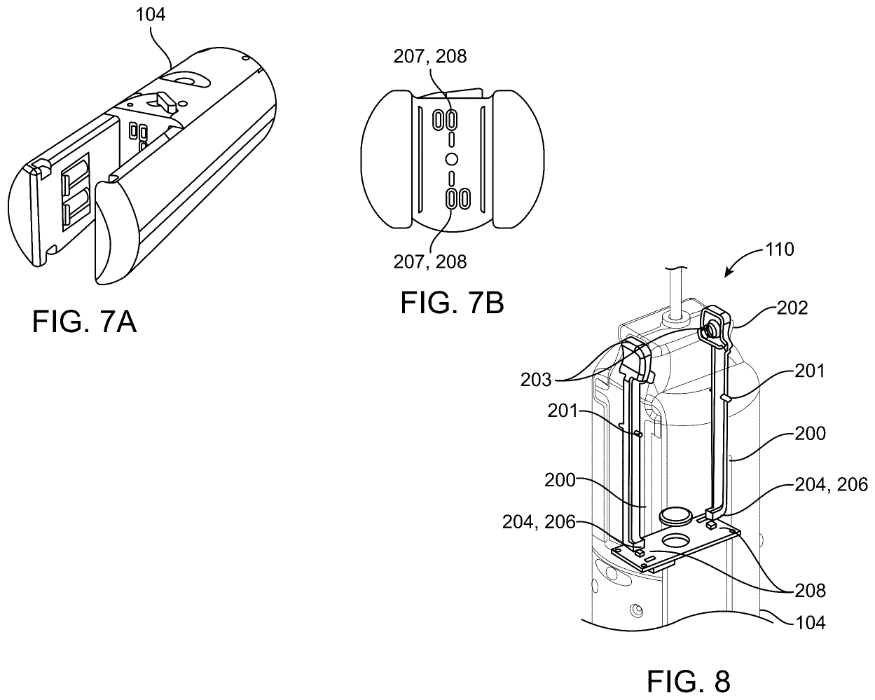

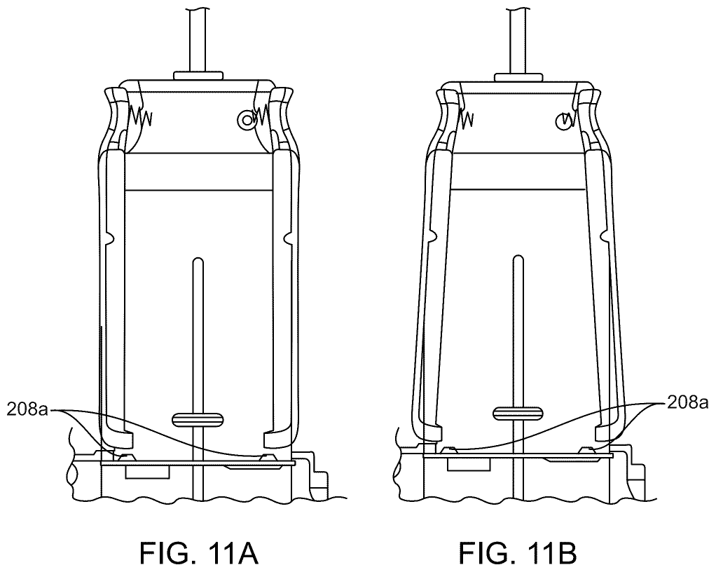

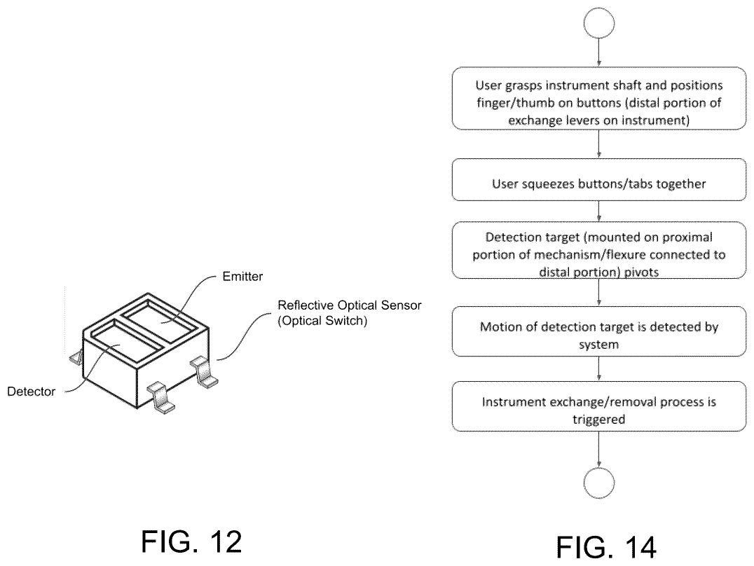

Electronic detectors 208 are positioned within the IDS. Each is positioned and intended to detect whether its corresponding detection target 206 has been displaced in the manner that results from a user pressing the proximal part 204 of the exchange lever 200. The types of detectors used for this purpose may vary. In some implementations such as the one shown in FIGS. 11A and 11B, an optical switch, such as an Onsemi/Fairchild QRE1113 Miniature Reflective Object Sensors is used as a detector to detect motion of the detector target, and/or to detect when the detector target is in alignment with the detector. See FIG. 12. For these configurations, the detector optical target may be a simple geometric feature that blocks the optical sensor, or it may have a reflective component (shiny material, reflective tape, marking, reflective paint, light color, etc.) that increases the light intensity difference between the first position and the second position (which corresponds to an instrument exchange request by a user). The detectors 208 are exposed through windows or openings 207 in the IDS housing (FIG. 7B) and the drape connector (FIG. 5B), allowing passage of light between the detectors and detection targets.

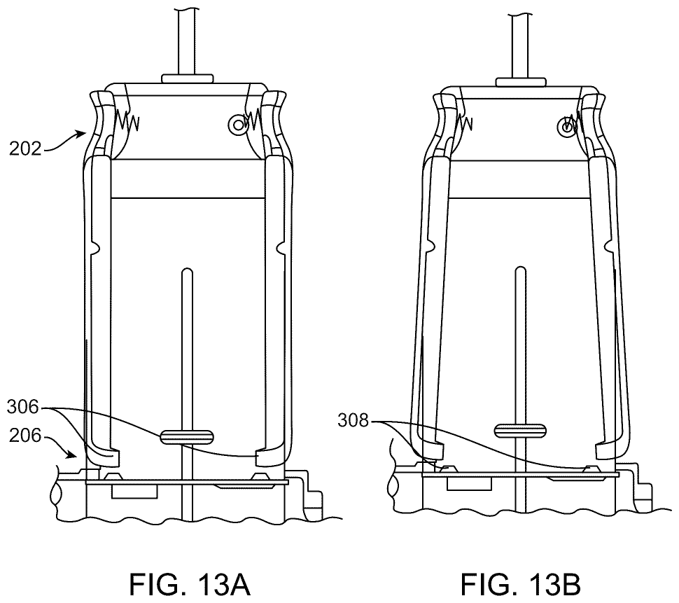

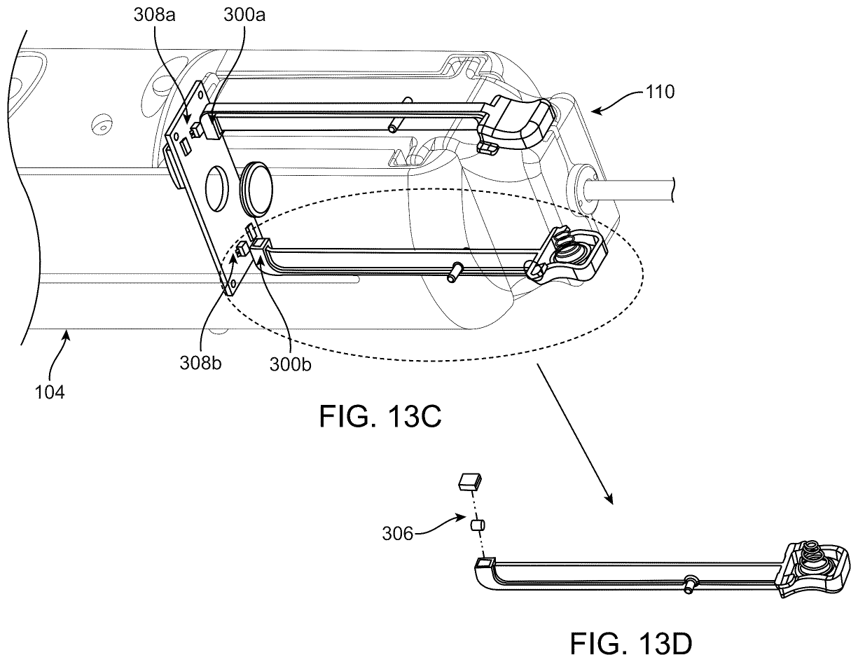

As shown in FIGS. 13A and 13B, an alternative configuration may be used in which the detector 308 is a Hall effector sensor, and the detection target includes a magnet 306 at the distal part 202 of the exchange lever. In this case, the detector detects the presence and magnitude of the magnet's magnetic field. This is illustrated in FIGS. 13A and 13B. This configuration is particularly beneficial because it can be used to tell the system what orientation the adapter is in. This can be important for control of the IDS, since the system must know the orientation of the surgical instrument within in adapter is needed so that the control algorithm for the IDS can map IDS actuators to their respective drive inputs on the instrument adapter. With this configuration, one of the exchange levers is a north pole lever 300a, with north pole polarity of the magnet at that lever positioned to be capable of detection by a hall sensor within the adapter-receiving region of the IDS. The other is a south pole lever 300b, with south pole polarity of the magnet e positioned to be capable of detection by a hall sensor within the adapter-receiving region of the IDS. In order to set orientation, the levers are assembled relative to the drive carriages/drive inputs within the IDS and/or to a distinct feature on the adapter box, e.g., flush port cap. FIG. 13D shows that the magnet may be enclosed within the proximal end of the exchange lever using a cap. When the adapter is sufficiently close to the IDS, the two hall sensors detect polarity of the two exchange levers. Therefore, orientation of the instrument adapter is determined using the readings of the corresponding hall sensors. For example, reading of north polarity by hall sensor 308a and south polarity by sensor 308 might be registered by the system as 0 degree orientation of the adapter, while reading of south polarity by hall sensor 308a and north polarity by sensor 308 might be registered by the system as 0 degree orientation of the adapter.

Each exchange lever preferably has at least a first position and a second position. In the first position, shown in FIGS. 8, 9, 10A, 11A and 12A, the distal part 202 is in its outermost position relative to the longitudinal axis of the adapter, and the proximal part 204 is in its innermost position relative to the longitudinal axis of the adapter. In the embodiments shown, in the first position the detection targets 206 are aligned with the detectors 208. In the second position, shown in FIGS. 10B, 11B and 12B, due to force against the press region of the distal part 202 by a user's fingers, the distal part 202 is displaced inwardly and the proximal part 204 is displaced outwardly, displacing the detection targets 206 away from, and out of alignment with, the detectors 208. Note that the term “aligned” in this context need not require direct alignment; it means that the position of the detection targets relative to the detectors is sufficient to generate signals by the detectors that are recognized by the system as indicating that the user is grasping the adapter. In some cases, such as with some optical sensors, this might be absence or presence of the detection targets, such as when optical sensors are blocked/unblocked by the detection targets, or when a Hall sensor detects the presence or absence of a magnetic field. In other cases, the response is threshold-based. For example, a threshold is set such that moving the detection targets out of alignment with the detectors drops the sensed criteria below the defined threshold, indicating that the user is grasping the adapter. Examples of thresholds include, without limitation: the amount of visible or infrared light/electromagnetic radiation reflected off the detection target and detected by an optical sensor; the magnitude of the magnetic field sensed by the Hall sensors; the distance between the detection target and detector based on the signals received from the detectors.

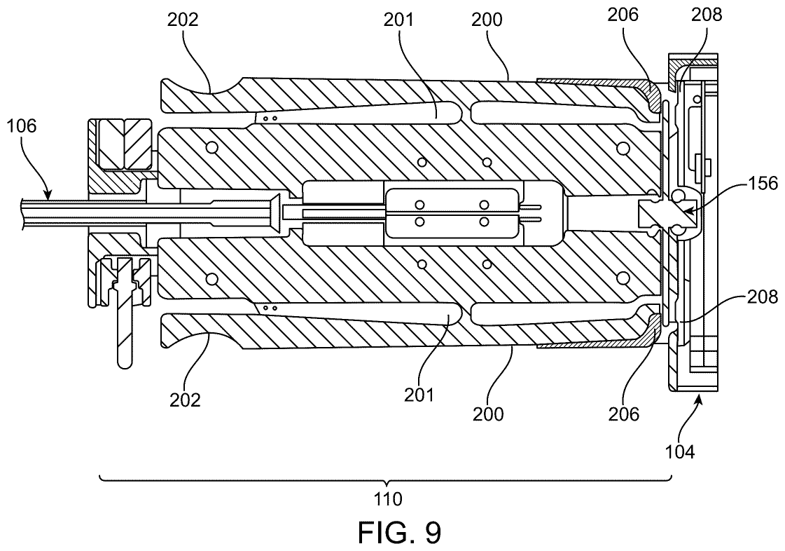

In the FIG. 8 embodiment, return springs bias the exchange lever in the first position so that when the user stops applying force to the press region, the exchange lever returns to the first position. In the FIG. 9 embodiment, the pivot geometry of the exchange lever is formed by a flexure, which combines both the pivoting motion and a return spring force. This return spring force returns the exchange lever to the first position on release of pressure applied to the press region.

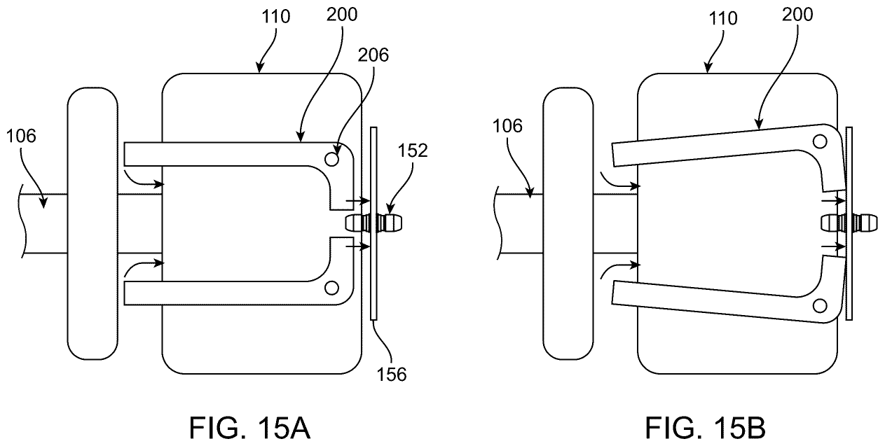

In some embodiments, the exchange input assembly plays the further role of helping to mechanically disengage the proximal end of the adapter from the drape connector IDS begins to open. More specifically, the exchange input assembly can be configured to include a cam portion that presses against the drape connector as the exchange input assembly pivots to the second position, disengaging the mating feature 152 of the drape connector from the female portion 150b at the proximal face of the adapter. See FIGS. 15A and 15B. This has the further advantage of pushing the drape connector against the IDS, ensuring that it and the drape remain in place on the IDS as the instrument is removed. This may be configured as a single stage process, where user pressure on the exchange input assembly performs a single action in which the detection targets are moved sufficiently to provide the needed input to the system confirming that the user is grasping the instrument while signaling the user's intent to remove the instrument, while simultaneously camming the adapter body away from the drape connector. For example, after the user movement of the detection targets results in the signals to the system (and, for example, the IDS begins to open), further squeezing of the distal parts of the exchange input assembly moves the proximal parts further to push the adapter off the retention features of the drape connector. The movement may be a smooth throw with no detents, or detents or other features may generate a tactile click, bump or increase in tension to let user know that first “half-press” has been accomplished. The full throw may then generate another tactile sensation using such detects or the naturally larger force felt by continued motion of the exchange assembly as it reaches full throw. In a variation of this embodiment, rather than a multi-stage throw of the exchange assembly, the exchange assembly may perform the same functions with multiple stages of the same throw. In alternative embodiments, the cam function can be performed using a mechanism independent of the exchange input assembly.

### Communicating Instrument Orientation

### Method

A method of informing the robotic system that the user wishes to remove the instrument, such as for an instrument exchange, will next be described with respect to FIG. 14. Control of the electronic functions associated with this method may be performed using the processor 30.

In an initial step, a user grasps the adapter, with the user's thumb on the distal part 202 of one exchange lever 200, and one or more of the user's opposed fingers on the distal part of the opposite exchange lever. The user squeezes the distal parts toward the adapter body, causing the proximal part 204 of the exchange lever to pivot outwardly.

The system receives signals from the detectors. When those signals indicate that the detection targets have moved out of alignment with the detectors (as described above), the system recognizes that the user is grasping the adapter. Accordingly, the system undertakes subsequent steps needed for instrument remove or exchange. These steps may include: activating motors in the IDS to cause it to open or electronically releasing a lock in the IDS allowing it to be manually opened. A non-limiting example of manually opening the IDS is found in the discussion of the lever/know 138 (see FIG. 4 of this application) in US 2021/169595. Other actions may additionally include causing the IDS to move the jaws of the surgical instrument to an open position if they are closed, straightening the instrument if it is articulated, or activating motors of the manipulator arm to cause the arm to retract in the z-axis direction (where the z-axis is coincident with the longitudinal axis of the instrument mounted to the IDS) so that the robotic manipulator retracts away from the adapter after the IDS has opened.

A particular sequence of events for exchanging an instrument might be as follows:
The user begins exchange sequence by grasping the instrument to depress the distal parts of the exchange assembly as described above, and the system recognizes that the user intends to remove the instrument and has a secure hand on the instrument.
The instrument jaw opens if it is not already open.
The system retracts the manipulator arm (along instrument shaft axis)
The arms of the IDS move to the open position.
The user removes the instrument, separating the drape from the drape connector if it has not already been separated by actuation of a camming feature.
The user inserts a new instrument into the IDS and presses it proximally to engage the retention features with those of the drape connector.
A button may be placed to cause the arms of the IDS to close, locking the instrument in place, or locking may be initiated by the detectors detecting the presence of the detection targets of the new instrument, or by an alternative instrument sensing feature.
The user hand-guides the instrument (which is attached to the IDS) to the position at which the previous instrument was disposed. For safety, the system may be configured to stop this motion just short of the previous working position of the previously used instrument in the body cavity, for safety. In some implementations, the system may undertake auto-inserting function initiated by a button press.

### Conclusion

Concepts described in this application encompass an instrument adapter for attachment of an instrument to a robotic surgical system which also contains a region or button for the user to depress, indicating a desire to initiate an instrument exchange. This button or region is located in a proximal position to the instrument shaft, passively constraining the user to maintain retention of the weight of the instrument. This proximal position allows the user to request an instrument exchange without shifting hand position.

Several advantages are provided by the concepts described here, these include the assurance that a user is grasping the instrument shaft in a stable position before the instrument is released from the IDS, and in some cases the function of making use of the mechanical advantage to additionally help to push the instrument off the drape connector and push the drape connector into the IDS, assuring retention. With the described embodiments, the user can both express an intent to exchange the instrument to the robotic surgical system as well as remove the instrument from the robotic surgical system without shifting hand position.

While certain embodiments have been described above, it should be understood that these embodiments are presented by way of example, and not limitation. It will be apparent to persons skilled in the relevant art that various changes in form and detail may be made therein without departing from the scope of the invention characterized by the claims. This is especially true in light of technology and terms within the relevant art(s) that may be later developed. Moreover, features of the various disclosed embodiments may be combined in various ways to produce various additional embodiments.

All patents, patent applications and printed publications referred to above, including for purposes of priority, are incorporated herein by reference.

We claim:

1. A method of removing a surgical instrument from a robotic manipulator, comprising:
providing a robotic manipulator having an expandable receiver for receiving a proximal end of a surgical instrument, the receiver having an open position in which a base of a surgical instrument may be introduced into the actuator assembly, and a closed position in which the base is releasably engaged by the receiver;
with the receiver in the closed position, grasping the surgical instrument using a hand of the user;
in response to the grasping of the surgical instrument, moving the receiver from the closed position to the open position; and
with the receiver in the open position, removing the surgical instrument from the receiver.

2. The method of claim 1, wherein the grasping step is performed using a single hand of the instrument.

3. The method of claim 1, wherein the method includes, during the grasping step, detecting displacement of detector targets on the instrument using detectors on the receiver.

4. The method of claim 3, wherein detecting displacement includes detecting displacement of a first detector target moveable in response to depression of a user thumb against a first portion of the surgical instrument, and detecting displacement of a second detector target moveable in response to depression of at least one user finger against a second portion of the surgical instrument, wherein the user thumb and user finger are on a common hand of the user.

5. The method of claim 3, wherein when the user grasps the instrument, the detector targets are moveable from a first position to a second position, and wherein in the second position the detectors generate a signal indicating that the detector targets are not in alignment with the corresponding detectors.

6. The method of claim 5, wherein in the second position the detectors generate a signal indicating at least one of loss of presence or decrease in proximity of the detector targets to the detectors.

7. The method of claim 1, wherein moving the receiver from the closed position to the open position comprises actuating a motor, actuation of the motor moving the receiver from the closed position to the open position.

8. The method of claim 1, wherein moving the receiver from the closed position to the open position comprises electronically releasing a lock, and then, after releasing the lock, manually moving the receiver from the closed position to the open position.

9. A robotic system assembly comprising:
a surgical instrument having an elongate shaft, an end effector at a distal end of the elongate shaft, and a proximal body on the proximal end of the elongate shaft;
an actuator assembly, the actuator assembly including a first arm and a second arm having opposed first and second faces defining an instrument-receiving portion between the first arm and the second arm, the actuator assembly having an open position in which the proximal body of the surgical instrument may be introduced into the instrument-receiving portion of the actuator assembly, and a closed position in which the proximal body is releasably engaged by the actuator assembly;
at least one detector target arranged on the proximal body such that manual grasping of the proximal body by a user displaces the detector target relative to the proximal body;
a least one sensor on the actuator assembly, said at least one sensor configured to detect displacement of the detector target relative to the proximal body when the proximal body is engaged by the actuator assembly in the closed position; and
at least one motor configured to move the actuator assembly from the closed position to the open position in response to detection of displacement of the detector target relative to the proximal body.

10. The robotic system assembly of claim 9, wherein the at least one detector target comprises a first detector target moveable in response to depression of a user thumb against a first portion of the proximal body, and a second detector target moveable in response to depression of at least one user finger against a second portion of the proximal body, wherein the user thumb and user finger are on a common hand of the user.

11. The robotic system assembly of claim 9, wherein the proximal body includes at least one member having a distal part and a proximal part, wherein the at least one detector target is disposed on the proximal part of the member, and wherein the proximal part is displaceable relative to the proximal body in response to manual depression of the distal part by a digit of a user.

12. The robotic system assembly of claim 10, wherein:
the proximal body includes a first member, wherein the first portion of the proximal body is a distal part of the first member and wherein the first detector is on a proximal part of the first member;
the proximal body includes a second member, wherein the second portion of the proximal body is a distal part of the second member and wherein the second detector is on a proximal part of the first member.

13. The robotic system assembly of claim 9, wherein the sensor is an optical sensor.
